(19) United States
(12) Patent Application Publication
(10) Pub. No.: US 2024/0376057 A1
(43) Pub. Date: Nov. 14, 2024

(54) 2,4-DIOXO-1,2,3,4-TETRAHYDROPYRIMIDINE DERIVATIVES AND THEIR USE IN THE TREATMENT OF TUMORS

(71) Applicants: ISTITUTO PASTEUR ITALIA, Roma (IT); IRCCS NEUROMED S.P.A., Pozzilli (IT)

(72) Inventor: Luigi FRATI, Roma (IT)

(21) Appl. No.: 18/687,454

(22) PCT Filed: Jun. 1, 2022
(86) PCT No.: PCT/IB2022/055143

§ 371 Date: Feb. 28, 2024

(30) Foreign Application Priority Data
Sep. 1, 2021 (IT) 102021000022682

(51) Int. Cl. C07D 239/557; A61K 31/513; A61P 35/00

(52) U.S. Cl. CPC C07D 239/557; A61K 31/513; A61P 35/00

(57) ABSTRACT

1,2,3,4-tetrahydropyrimidine-5-carboxamide derivatives of formula (I) as aerobic glycolysis inhibitors and their use in the treatment of tumors.

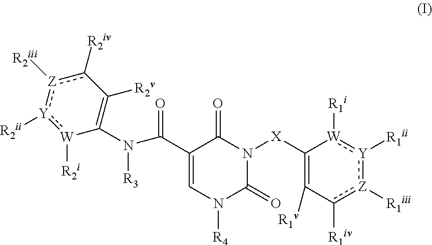

### FIELD OF THE INVENTION

The present invention relates to pyrimidine derivatives and their use in the treatment of tumors.

### BACKGROUND OF THE INVENTION

Classical therapies against tumors currently involve surgery, radiotherapy, chemotherapy, immunological procedures and gene therapy.

It is worth noting that neoplastic cells need both nutrients and oxygen for their growth; recently the attention was pointed on the inhibition of neovascularization to starve the neoplastic cells and stop the progression of the tumors (Goldman E, The growth of malignant disease in man and the lower animals with special reference to the vascular system Lancet (1907) 2:1236-40; Gullino P M, Angiogenesis and oncogenesis J Natl Cancer Inst (1978) 61:639-43; Ferrara N, VEGF and the quest for tumor angiogenesis factors Nature Rev Cancer (2002) 2:795-803).

Normal and tumor cells need for their replication energy, which is accumulated in the cytoplasm as adenosyntriphosphate (ATP) molecules, produced through the metabolism of glucose, which is first bi-phosphorylated, and then processed to enter into the mitochondrial Krebs cycle (energetic way called “oxidative phosphorylation”) with a consumption of oxygen and a final production of both CO₂ and 38 ATP energetic molecules (36 ATP molecules net production).

In cancer cells Otto Warburg described an alternative energetic pathway called “aerobic glycolysis” (Warburg O, The metabolism of tumors J Physiol Chem (1910) 56:66-305). Glycolysis is a process by which cells convert glucose into pyruvate and lactate to generate ATP (4 molecules, net production of 2). Warburg received in 1931 the Nobel prize “for his discovery of the nature and mode of action of the respiratory enzymes” in cancer cells. Warburg's opinion was that the aerobic glycolysis is “causative” of cancer (Warburg O, On the origin of cancer cells Science (1956) 123:309-14), whereas today we know that the Warburg's metabolic shift (aerobic glycolysis) is secondary to the multiple oncogenic steps, which modify the metabolism, i.e. the epigenetic regulation of the activity of many enzymes, such as pyruvate kinase-PK and lactate dehydrogenase (Cairns R A, Harris I S, Mak T W, Regulation of cancer cell metabolism Nat Rev Cancer (2011) 11(2):85-95).

In search of new ways to be used in tumor therapy the interest about the cancer aerobic glycolysis is today reinforced (Kritikou E, Warburg effect revisited Nature Reviews Cancer (2008) 8:247; Xu X D, Shao S X, Jiang H P et al, Warburg effect or reverse Warburg effect? A review of cancer metabolism Oncology Research and Treatment (2015) 38:117-122). The aim is to search for molecules which inhibit such a metabolic pathway and may be used to stop the cancer growth. The key enzyme is lactate dehydrogenase (LDH), which catalyzes the passage pyruvate-lactate with a net production of 2 energetic molecules of ATP with a parallel reduction of nicotinamide adenine dinucleotide (NAD) into NADH.

Unfortunately, inhibitors of LDH activity, such as oxamate, exert their effect only at high (mM) concentrations. Although aerobic glycolysis is the subject of intensive investigation, to date no compounds capable of inhibiting this metabolic pathway at a low (μM) concentration that can be used as antitumor drugs in humans have been identified.

### SUMMARY OF THE INVENTION

The object of this disclosure is to provide novel antitumor drugs able to reduce tumor growth through inhibition of aerobic glycolysis, preferably through the inhibition of the lactate dehydrogenase enzyme.

According to the invention, the above object is achieved thanks to the subject matter recalled specifically in the ensuing claims, which are understood as forming an integral part of this disclosure.

The present invention relates to pyrimidine derivatives, specifically 1,2,3,4-tetrahydropyrimidine-5-carboxamide derivatives and bioisosteric derivatives thereof, and their use in the treatment of tumors. The inventor has in fact discovered that 1,2,3,4-tetrahydropyrimidine-5-carboxamide derivatives are inhibitors of aerobic glycolysis through the inhibition of the lactate dehydrogenase enzyme.

In one embodiment, the present invention concerns a 1,2,3,4-tetrahydropyrimidine-5-carboxamide derivative of formula (I):

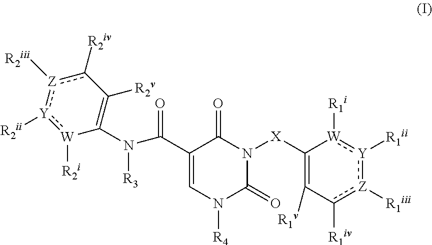

wherein
R₁ⁱ, R₁ⁱⁱ, R₁ⁱⁱⁱ, R₁^{v}, R₂ⁱ, R₂ⁱⁱ, R₂ⁱⁱⁱ, R₂^{v} are independently selected from: none, hydrogen, C₁₋₆ alkyl, C₃₋₁₀ cycloalkyl, C₂₋₆ alkenyl, C₂₋₆ alkynyl, C₁₋₆ alkoxy, C₂₋₆ alkoxycarbonyl, 3-10 member heterocycloalkyl, halogen, CN, NH₂, OH, NO₂, OR₇, NHR₈, NR₇R₈, and NHCOR₇;
R₃ and R₄ are independently selected from: hydrogen, C₁₋₆ alkyl, C₃₋₁₀ cycloalkyl, C₂₋₆ alkenyl, C₂₋₆ alkynyl, C₁₋₆ alkoxy, C₂₋₆ alkoxycarbonyl, 3-10-member heterocycloalkyl, halogen, CN, NH₂, OH, and NO₂;
R₇ and R₈ are independently selected from: C₁₋₆ alkyl, C₃₋₁₀ cycloalkyl, C₂₋₆ alkenyl, C₂₋₆ alkynyl, C₁₋₆ alkoxy, C₂₋₆ alkoxycarbonyl, 3-10 member heterocycloalkyl, and a substituted or unsubstituted aromatic ring Ar₁;
X is selected from: CH₂, C(═O), S, SO₂, SO, and none; and
W, Y and Z are independently selected from: C, N, C(═O), C(═S);
a pharmaceutically acceptable salt, hydrated salt, polymorph, racemate, diastereoisomer or enantiomer thereof,
with the exception of:
2,4-Dioxo-3-(2-fluorobenzyl)-N-phenyl-1,2,3,4-tetrahydropyrimidine-5-carboxamide,
2,4-Dioxo-3-(2-chlorobenzyl)-N-phenyl-1,2,3,4-tetrahydropyrimidine-5-carboxamide,
2,4-Dioxo-3-(2-fluorobenzyl)-N-(4-fluorophenyl)-1,2,3,4-tetrahydropyrimidine-5-carboxamide,
2,4-Dioxo-3-(2-fluorobenzyl)-N-(4-chlorophenyl)-1,2,3,4-tetrahydropyrimidine-5-carboxamide,
2,4-Dioxo-3-(2-fluorobenzyl)-N-(4-methylphenyl)-1,2,3,4-tetrahydropyrimidine-5-carboxamide,
2,4-Dioxo-3-(2-fluorobenzyl)-N-(pyridin-4-yl)-1,2,3,4-tetrahydropyrimidine-5-carboxamide,
2,4-Dioxo-3-(2-fluorobenzyl)-N-(pyridin-2-yl)-1,2,3,4-tetrahydropyrimidine-5-carboxamide,
2,4-Dioxo-3-(2-fluorobenzyl)-N-(thiazol-2-yl)-1,2,3,4-tetrahydropyrimidine-5-carboxamide.

In one embodiment, the present invention concerns a 1,2,3,4-tetrahydropyrimidine-5-carboxamide derivative of formula (I) for use in the treatment and/or prevention of a tumor and a pharmaceutical composition comprising at least one 1,2,3,4-tetrahydropyrimidine-5-carboxamide derivative of formula (I) and a pharmaceutically acceptable vehicle.

### BRIEF DESCRIPTION OF THE DRAWINGS

The invention will now be described in detail, purely by way of illustrative and non-limiting example, with reference to the attached figures, wherein:

FIG. 1: Effect of oxamate on proliferation of medulloblastoma tumor cells in vitro (doses between 1 and 100 mM). ns=not significant, **p<0.01, ***p<0.001 by ANOVA test through GraphPad Prism version 6.0.

FIG. 2: Effect of compound #1 at millimolar doses on tumor cell proliferation. ***p<0.001 by ANOVA test through GraphPad Prism version 6.0.

FIG. 3: Panel A. Effect of compound #1 at micromolar doses on tumor cell proliferation. ns=not significant, ***p<0.001 by ANOVA test through GraphPad Prism version 6.0. Panel B. Dose-response curve of compound #1 generated by non-linear regression.

FIG. 4: Effect of compound #1 on the activity of LDH enzyme.

FIG. 5: LDHA gene expression in human pancreatic cancer.

FIG. 6: Panel A. Effect of compound #1 on tumor cell proliferation. ns=not significant, * p<0.05, *** p<0.001 by ANOVA test through GraphPad Prism version 6.0. Panel B. Dose-response curve of compound #1 generated by non-linear regression.

FIG. 7: Effect of compound #1 on tumor growth of pancreatic tumor cells in vivo. Panel A. Effect of compound #1 on tumor growth. ns=not significant, ** p<0.01 by t-test through GraphPad Prism version 6.0. Panel B. Variation of mouse weight at T=0 and T=17. Panel C. Photographs of xenografted mice at T=17 treated with vehicle and compound #1.

FIG. 8: In vivo luminescence of tumor masses in mice treated with compound #1 or vehicle.

FIG. 9: Dose-response effect of the various compounds (#1-#7) on tumor cell proliferation. Cells were treated with the indicated concentration of the compounds for 24 hrs and then counted. Values on the Y axis represent the percentage of cell proliferation.

### DETAILED DESCRIPTION OF THE INVENTION

In the following description, numerous specific details are given to provide a thorough understanding of embodiments. The embodiments can be practiced without one or more of the specific details, or with other methods, components, materials, etc. In other instances, well-known structures, materials, or operations are not shown or described in detail to avoid obscuring aspects of the embodiments.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The headings provided herein are for convenience only and do not interpret the scope or meaning of the embodiments.

The term “1,2,3,4-tetrahydropyrimidine-5-carboxamide derivative” means compounds derived from the following chemical structure:

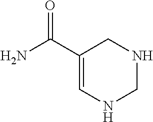

The term “aerobic glycolysis inhibitor” is understood to mean a compound that inhibits the aerobic glycolysis pathway, preferably inhibits the lactate dehydrogenase (LDH) enzyme.

The term “treatment” is understood to mean prevention or alleviation of symptoms or condition, or inhibition of the advancement of a pathology that includes cell proliferation disorders (including tumor and antitumor drug-resistant tumor).

The term “C₁₋₆ alkyl” is understood as a chain of 1-6 carbon atoms containing only single bonds between adjacent carbon atoms. Preferably, the C₁₋₆ alkyl group is selected from methyl, ethyl, propyl, iso-propyl, butyl and its isomers, pentyl and its isomers, hexyl and its isomers.

The term “C₂₋₆ alkenyl” is understood as a chain of 2-6 carbon atoms containing at least one double bond between adjacent carbon atoms. Alkenyls include cis and trans isomers. Preferably, the C₂₋₆ alkenyl group is selected from ethylenyl, propylenyl, 1-butenyl, 2-butenyl, isobutenyl, 1-pentenyl, 2-pentenyl, 3-methyl-1-butenyl, 2-methyl-2-butenyl, 2,3-dimethyl-2-butenyl.

The term “C₂₋₆ alkynyl” is understood as a 2-6 carbon atom chain containing at least one triple bond between adjacent carbon atoms. Preferably, the C₂₋₆ alkynyl group is selected from ethynyl, propynyl, pentynyl, and their isomers.

The term “C₁₋₆ alkoxy” is understood as 1-6 carbon atom alkyl chains containing one oxygen atom. Preferably, the C₁₋₆ alkoxy group is selected from methoxy, ethoxy, propoxy, pentoxy and their isomers.

The term “C₂₋₆ alkoxycarbonyl” is understood as a 2-6-atom alkyl chain containing an OC═O group. Preferably, the C₂₋₆ alkoxycarbonyl group is selected from methoxycarbonyl, ethoxycarbonyl, methoxycarbonyl methyl, ethoxycarbonyl methyl.

The term “C₃₋₁₀ cycloalkyl” is understood as a cyclic hydrocarbon group having 3-10 carbon atoms. Each carbon atom can be substituted with one or more substituents selected from C₁₋₆-alkyl or halogen. Preferably, the C₃₋₁₀ cycloalkyl group is selected from cyclopropyl, cyclobutyl, cyclopentyl, and cyclohexyl.

The term “3-10 member heterocycloalkyl” is understood to mean a saturated cyclic hydrocarbon having 3 to 10 carbon atoms, wherein one or more carbon atoms are substituted by one or more heteroatoms, as nitrogen, sulfur, oxygen, or combinations thereof, for example, but not limited to, a nitrogen atom and a sulfur atom, a nitrogen atom and an oxygen atom. Preferably, the 3-10-member heterocycloalkyl group is selected from pyrrolidin, piperidine, piperazine, morpholine, thiomorpholine.

The term “heterocycle” is understood as aromatic heterocyclic compounds in which one and more carbon atom(s) of the molecule's skeleton has been replaced by an atom other than carbon. Typical heteroatoms include nitrogen, oxygen, and sulfur. Preferably, the heterocycle is selected from pyrrole, thiophene, furan, pyridine, and pyrimidine.

The term “benzofused heterocycle” is understood as an aromatic heterocyclic compound in which one or more bond(s) are fused with a phenyl ring. Preferably, the benzofused heterocycle is selected from indole, benzothiophene, benzofuran, quinoline, and isoquinoline.

The term “pharmaceutically acceptable salt or hydrated salt” refers to salts with inorganic and organic acids, or salts with inorganic or organic bases, that retain their original biological properties and do not show undesirable side effects. Salts with inorganic acids are prepared using acids such as, for example, hydrochloric acid, sulfuric acid, phosphoric acid, nitric acid. Salts with organic acids are prepared using acids such as, for example, acetic acid, propionic acid, succinic acid, maleic acid, citric acid, ascorbic acid, pamoic acid, glycolic acid, stearic acid, phenylacetic acid, benzoic acid, salicylic acid, methanesulfonic acid, toluenesulfonic acid. Salts with inorganic bases are prepared using bases such as, for example, sodium carbonate, caustic soda, ammonia, ammonium hydroxide. Salts with organic bases are prepared using bases such as, for example, dimethylamine, piperidine, piperazine, morpholine, thiomorpholine.

The inhibition of the lactate dehydrogenase activity lowers the progression of tumors (loss of energetic ATP molecules and precursors for the biosynthesis of macromolecules), with a therapeutic application especially in tumors with high glycolysis, such as brain and pancreatic tumors (Le A, Cooper C R, Gouw A M et al, Inhibition of lactate dehydrogenase A induces oxidative stress and inhibits tumor progression Proc Natl Acad Sci USA (2010) 107:2037-42; Calvaresi E C, Granchi C, Tuccinardi T et al. Dual targeting of the Warburg effect with a glucose-conjugated lactate dehydrogenase inhibitor ChemBioChem (2013) 14, issue 17; Rong Y, WuW, Ni X et al. Lactate dehydrogenase A is overexpressed in pancreatic cancer and promotes the growth of pancreatic cancer cells Tumor Biol (2013) 34:1523-30; Valvona C J, Fillmore H L, Nunn P B, Pilkington G J. The regulation and function of lactate dehydrogenase A: therapeutic potential in brain tumor Brain Pathol (2016) 26:1-17).

A recent review has summarized the data about LDH different expression in various cancer cell lines to search inhibitors of LDH activity to be used in antiblastic therapy: none of these molecules has been registered for therapeutic application (Feng Y et al, Lactate dehydrogenase A. A key player in carcinogenesis and potential target in cancer therapy Cancer Med (2018) 7:6124-36; Le A, Cooper C R, Gouw A M et al, Inhibition of lactate dehydrogenase A induces oxidative stress and inhibits tumor progression Proc Natl Acad Sci USA (2010) 107:2037-42; Calvaresi E C, Granchi C, Tuccinardi T et al. Dual targeting of the Warburg effect with a glucose-conjugated lactate dehydrogenase inhibitor ChemBioChem (2013) 14, issue 17).

10 years ago, during research about the induction of apoptosis on human neuroblastoma cells in vitro (Petroni M, . . . Frati L, . . . Myc sensitizes human neuroblastoma to apoptosis . . . Molecular Cancer Res (2011) 9:67-77) the present inventor observed that these tumor cells exert aerobic glycolysis, with the lactate dehydrogenase enzyme activity inhibited by some well-known molecules, i.e. oxamate or bromopyruvate (spectrophotometric assay). Unfortunately, these molecules—despite very effective in inhibiting LDH—exert the activity only at high (mM) concentrations or have the burden of toxic effects that prevent their use in a clinical treatment.

The present invention concerns 1,2,3,4-tetrahydropyrimidine-5-carboxamide derivatives of formula (I) and their bioisosteric derivatives, and their use for the treatment of a patient suffering from a tumor. The inventor has in fact discovered that 1,2,3,4-tetrahydropyrimidine-5-carboxamide derivatives of formula (I) are inhibitors of aerobic glycolysis, namely inhibitors of lactate dehydrogenase.

In one embodiment, the present invention concerns a 1,2,3,4-tetrahydropyrimidine-5-carboxamide derivative of formula (I):

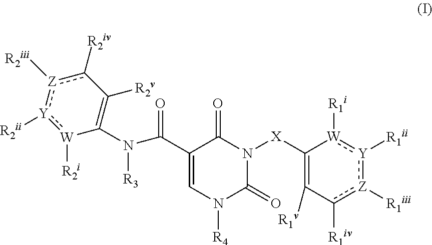

wherein
R₁ⁱ, R₁ⁱⁱ, R₁ⁱⁱⁱ, R₁^{v}, R₂ⁱ, R₂ⁱⁱ, R₂ⁱⁱⁱ, R₂^{v} are independently selected from: none, hydrogen, C₁₋₆ alkyl, C₃₋₁₀ cycloalkyl, C₂₋₆ alkenyl, C₂₋₆ alkynyl, C₁₋₆ alkoxy, C₂₋₆ alkoxycarbonyl, 3-10 member heterocycloalkyl, halogen, —CN, —NH₂, —OH, —NO₂, —OR₇, —NHR₈, —NR₇R₈, and —NHCOR₇;
R₃ and R₄ are independently selected from: hydrogen, C₁₋₆ alkyl, C₃₋₁₀ cycloalkyl, C₂₋₆ alkenyl, C₂₋₆ alkynyl, C₁₋₆ alkoxy, C₂₋₆ alkoxycarbonyl, 3-10-member heterocycloalkyl, halogen, —CN, —NH₂, —OH, and —NO₂;
R₇ and R₈ are independently selected from: C₁₋₆ alkyl, C₃₋₁₀ cycloalkyl, C₂₋₆ alkenyl, C₂₋₆ alkynyl, C₁₋₆ alkoxy, C₂₋₆ alkoxycarbonyl, 3-10 member heterocycloalkyl, and a substituted or unsubstituted aromatic ring Ar₁;
X is selected from: —CH₂, —C(═O), —S, —SO₂, —SO, and none; and
W, Y and Z are independently selected from: —C, —N, —C(═O), —C(═S); a pharmaceutically acceptable salt, hydrated salt, polymorph, racemate, diastereoisomer or enantiomer thereof,
with the exception of:
2,4-Dioxo-3-(2-fluorobenzyl)-N-phenyl-1,2,3,4-tetrahydropyrimidine-5-carboxamide (1),
2,4-Dioxo-3-(2-chlorobenzyl)-N-phenyl-1,2,3,4-tetrahydropyrimidine-5-carboxamide (9),
2,4-Dioxo-3-(2-fluorobenzyl)-N-(4-fluorophenyl)-1,2,3,4-tetrahydropyrimidine-5-carboxamide (15),
2,4-Dioxo-3-(2-fluorobenzyl)-N-(4-chlorophenyl)-1,2,3,4-tetrahydropyrimidine-5-carboxamide (19),
2,4-Dioxo-3-(2-fluorobenzyl)-N-(4-methylphenyl)-1,2,3,4-tetrahydropyrimidine-5-carboxamide (20),
2,4-Dioxo-3-(2-fluorobenzyl)-N-(pyridin-4-yl)-1,2,3,4-tetrahydropyrimidine-5-carboxamide (22),
2,4-Dioxo-3-(2-fluorobenzyl)-N-(pyridin-2-yl)-1,2,3,4-tetrahydropyrimidine-5-carboxamide (23),
2,4-Dioxo-3-(2-fluorobenzyl)-N-(thiazol-2-yl)-1,2,3,4-tetrahydropyrimidine-5-carboxamide (24).

In one embodiment, R₁ⁱ, R₁ⁱⁱ, R₁ⁱⁱⁱ, R₁^{v}, R₂ⁱ, R₂ⁱⁱ, R₂ⁱⁱⁱ, R₂^{v}, R₃, and R₄ are independently selected from: hydrogen, C₁₋₆ alkyl, halogen.

In one embodiment, R₃ and R₄ are independently selected from: hydrogen, C₁₋₆ alkyl, and C₃₋₁₀ cycloalkyl.

In one embodiment, R₃ and R₄ are hydrogen.

In one embodiment, R₇ and R₈ are independently selected from: C₁₋₆ alkyl, and a substituted or unsubstituted aromatic ring Ar₁.

In one embodiment, X is selected from: CH₂, C(═O).

In one embodiment, W, Y and Z are C.

In one embodiment, the aromatic ring Ar₁ is selected from: phenyl, benzyl, phenoxy, benzyloxy, naphthyl, naphthyloxy, biphenyl, and heterocycle. Preferably, the aromatic ring Ar₁ is selected from: phenyl, benzyl, heterocycle.

In one embodiment, the one or more substituents of the aromatic ring Ar₁ are independently selected from: C₁₋₆ alkyl, C₃₋₁₀ cycloalkyl, C₂₋₆ alkenyl, C₂-s alkynyl, C₁₋₆ alkoxy, C₂₋₆ alkoxycarbonyl, 3-10 member heterocycloalkyl, halogen, CN, NH₂, OH, and NO₂. Preferably, the one or more substituents of the aromatic ring Ar₁ are independently selected from: C₁₋₆ alkyl, and halogen.

In one embodiment, the 1,2,3,4-tetrahydropyrimidine derivative of formula (I) is selected from:
2,4-Dioxo-3-benzyl-N-(3-chlorophenyl)-1,2,3,4-tetrahydropyrimidine-5-carboxamide (2);
2,4-Dioxo-3-(4-fluorobenzyl)-N-(2-fluorophenyl)-1,2,3,4-tetrahydropyrimidine-5-carboxamide (3);
2,4-Dioxo-3-benzyl-N-(2-chlorophenyl)-1,2,3,4-tetrahydropyrimidine-5-carboxamide (4);
2,4-Dioxo-3-benzyl-N-(3,5-dimethoxyphenyl)-1,2,3,4-tetrahydropyrimidine-5-carboxamide (5);
2,4-Dioxo-3-benzyl-N-(4-carboxamidophenyl)-1,2,3,4-tetrahydropyrimidine-5-carboxamide (6);
2,4-Dioxo-3-(4-methoxybenzyl)-N-(2-chlorophenyl)-1,2,3,4-tetrahydropyrimidine-5-carboxamide (7);
2,4-Dioxo-3-(2,4-difluorobenzyl)-N-phenyl-1,2,3,4-tetrahydropyrimidine-5-carboxamide (8);
2,4-Dioxo-3-(4-chlorobenzyl)-N-phenyl-1,2,3,4-tetrahydropyrimidine-5-carboxamide (10);
2,4-Dioxo-3-(2,4-dichlorobenzyl)-N-phenyl-1,2,3,4-tetrahydropyrimidine-5-carboxamide (11);
2,4-Dioxo-3-(3-fluoropyridin-4-ylmethyl)-N-phenyl-1,2,3,4-tetrahydropyrimidine-5-carboxamide (12);
2,4-Dioxo-3-(pyridin-4-ilmethyl)-N-phenyl-1,2,3,4-tetrahydropyrimidine-5-carboxamide (13);
2,4-Dioxo-3-(pyrimidin-4-ilmethyl)-N-phenyl-1,2,3,4-tetrahydropyrimidine-5-carboxamide (14);
2,4-Dioxo-3-(4-fluorobenzyl)-N-(4-fluorophenyl)-1,2,3,4-tetrahydropyrimidine-5-carboxamide (16);
2,4-Dioxo-3-(2,4-difluorobenzyl)-N-(4-fluorophenyl)-1,2,3,4-tetrahydropyrimidine-5-carboxamide (17);
2,4-Dioxo-3-(benzyl)-N-(2-chlorophenyl)-1,2,3,4-tetrahydropyrimidine-5-carboxamide (18);
2,4-Dioxo-3-(2-fluorobenzyl)-N-(4-aminophenyl)-1,2,3,4-tetrahydropyrimidine-5-carboxamide (21);
2,4-Dioxo-3-(benzyl)-N-(3,5-dichlorophenyl)-1,2,3,4-tetrahydropyrimidine-5-carboxamide (25).

The chemical structure of compounds (1) to (7) are shown below:

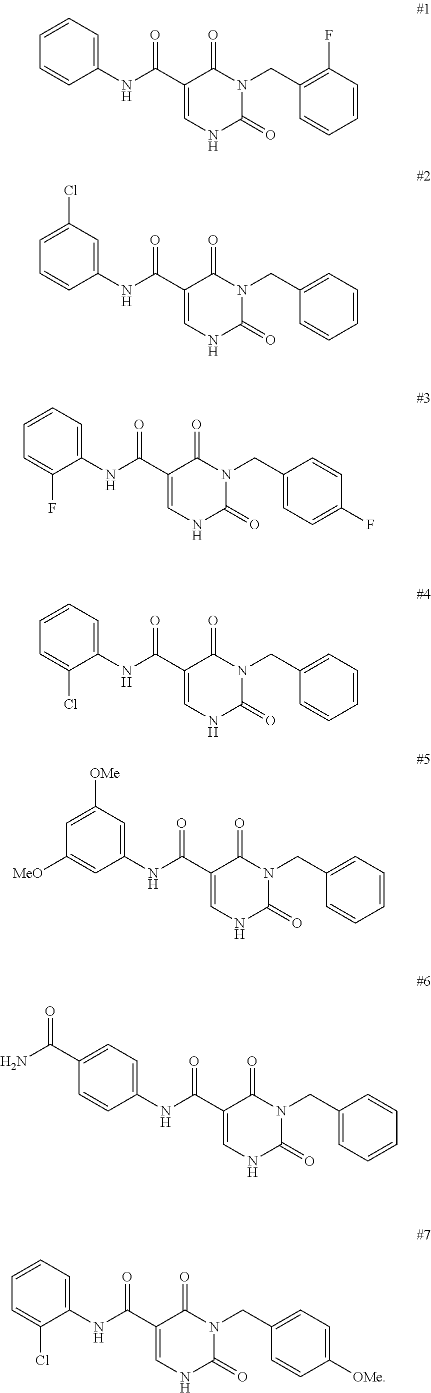

In an embodiment, the present invention concerns a 1,2,3,4-tetrahydropyrimidine-5-carboxamide derivative of formula (I) (as defined above) for use in the treatment of a tumor.

In an embodiment, the 1,2,3,4-tetrahydropyrimidine-5-carboxamide derivative of formula (I) for use in the treatment of a tumor is selected from:
2,4-dioxo-N-phenyl-3-(2-fluorobenzyl)-1,2,3,4-tetrahydropyrimidine-5-carboxamide (1);
2,4-Dioxo-3-benzyl-N-(3-chlorophenyl)-1,2,3,4-tetrahydropyrimidine-5-carboxamide (2);
2,4-Dioxo-3-(4-fluorobenzyl)-N-(2-fluorophenyl)-1,2,3,4-tetrahydropyrimidine-5-carboxamide (3);
2,4-Dioxo-3-benzyl-N-(2-chlorophenyl)-1,2,3,4-tetrahydropyrimidine-5-carboxamide (4);
2,4-Dioxo-3-benzyl-N-(3,5-dimethoxyphenyl)-1,2,3,4-tetrahydropyrimidine-5-carboxamide (5);
2,4-Dioxo-3-benzyl-N-(4-carboxamidophenyl)-1,2,3,4-tetrahydropyrimidine-5-carboxamide (6);
2,4-Dioxo-3-(4-methoxybenzyl)-N-(2-chlorophenyl)-1,2,3,4-tetrahydropyrimidine-5-carboxamide (7);
2,4-Dioxo-3-(2,4-difluorobenzyl)-N-phenyl-1,2,3,4-tetrahydropyrimidine-5-carboxamide (8);
2,4-Dioxo-3-(2-chlorobenzyl)-N-phenyl-1,2,3,4-tetrahydropyrimidine-5-carboxamide (9);
2,4-Dioxo-3-(4-chlorobenzyl)-N-phenyl-1,2,3,4-tetrahydropyrimidine-5-carboxamide (10);
2,4-Dioxo-3-(2,4-dichlorobenzyl)-N-phenyl-1,2,3,4-tetrahydropyrimidine-5-carboxamide (11);
2,4-Dioxo-3-(3-fluoropyridin-4-ylmethyl)-N-phenyl-1,2,3,4-tetrahydropyrimidine-5-carboxamide (12);
2,4-Dioxo-3-(pyridin-4-ilmethyl)-N-phenyl-1,2,3,4-tetrahydropyrimidine-5-carboxamide (13);
2,4-Dioxo-3-(pyrimidin-4-ilmethyl)-N-phenyl-1,2,3,4-tetrahydropyrimidine-5-carboxamide (14);
2,4-Dioxo-3-(2-fluorobenzyl)-N-(4-fluorophenyl)-1,2,3,4-tetrahydropyrimidine-5-carboxamide (15);
2,4-Dioxo-3-(4-fluorobenzyl)-N-(4-fluorophenyl)-1,2,3,4-tetrahydropyrimidine-5-carboxamide (16);
2,4-Dioxo-3-(2,4-difluorobenzyl)-N-(4-fluorophenyl)-1,2,3,4-tetrahydropyrimidine-5-carboxamide (17);
2,4-Dioxo-3-(benzyl)-N-(2-chlorophenyl)-1,2,3,4-tetrahydropyrimidine-5-carboxamide (18);
2,4-Dioxo-3-(2-fluorobenzyl)-N-(4-chlorophenyl)-1,2,3,4-tetrahydropyrimidine-5-carboxamide (19);
2,4-Dioxo-3-(2-fluorobenzyl)-N-(4-methylphenyl)-1,2,3,4-tetrahydropyrimidine-5-carboxamide (20);
2,4-Dioxo-3-(2-fluorobenzyl)-N-(4-aminophenyl)-1,2,3,4-tetrahydropyrimidine-5-carboxamide (21);
2,4-Dioxo-3-(2-fluorobenzyl)-N-(pyridin-4-yl)-1,2,3,4-tetrahydropyrimidine-5-carboxamide (22);
2,4-Dioxo-3-(2-fluorobenzyl)-N-(pyridin-2-yl)-1,2,3,4-tetrahydropyrimidine-5-carboxamide (23);
2,4-Dioxo-3-(2-fluorobenzyl)-N-(thiazol-2-yl)-1,2,3,4-tetrahydropyrimidine-5-carboxamide (24)
2,4-Dioxo-3-(benzyl)-N-(3,5-dichlorophenyl)-1,2,3,4-tetrahydropyrimidine-5-carboxamide (25).

In a preferred embodiment, the 1,2,3,4-tetrahydropyrimidine-5-carboxamide derivative of formula (I) for use in the treatment of a tumor is selected from:
2,4-dioxo-N-phenyl-3-(2-fluorobenzyl)-1,2,3,4-tetrahydropyrimidine-5-carboxamide (1);
2,4-Dioxo-3-benzyl-N-(3-chlorophenyl)-1,2,3,4-tetrahydropyrimidine-5-carboxamide (2);
2,4-Dioxo-3-(4-fluorobenzyl)-N-(2-fluorophenyl)-1,2,3,4-tetrahydropyrimidine-5-carboxamide (3);
2,4-Dioxo-3-benzyl-N-(2-chlorophenyl)-1,2,3,4-tetrahydropyrimidine-5-carboxamide (4);
2,4-Dioxo-3-benzyl-N-(3,5-dimethoxyphenyl)-1,2,3,4-tetrahydropyrimidine-5-carboxamide (5);
2,4-Dioxo-3-benzyl-N-(4-carboxamidophenyl)-1,2,3,4-tetrahydropyrimidine-5-carboxamide (6);
2,4-Dioxo-3-(4-methoxybenzyl)-N-(2-chlorophenyl)-1,2,3,4-tetrahydropyrimidine-5-carboxamide (7).

In a preferred embodiment, the 1,2,3,4-tetrahydropyrimidine-5-carboxamide derivative of formula (I) for use in the treatment of a tumor is selected from: 2,4-dioxo-N-phenyl-3-(2-fluorobenzyl)-1,2,3,4-tetrahydropyrimidine-5-carboxamide (1); 2,4-Dioxo-3-benzyl-N-(2-chlorophenyl)-1,2,3,4-tetrahydropyrimidine-5-carboxamide (4); 2,4-Dioxo-3-benzyl-N-(4-carboxamidophenyl)-1,2,3,4-tetrahydropyrimidine-5-carboxamide (6); 2,4-Dioxo-3-(4-methoxybenzyl)-N-(2-chlorophenyl)-1,2,3,4-tetrahydropyrimidine-5-carboxamide (7). More preferably, the 1,2,3,4-tetrahydropyrimidine-5-carboxamide derivative of formula (I) for use in the treatment of a tumor is 2,4-dioxo-N-phenyl-3-(2-fluorobenzyl)-1,2,3,4-tetrahydropyrimidine-5-carboxamide (1).

In an embodiment, the tumor is selected from brain and pancreatic tumors. Preferably, the tumor is selected from medulloblastoma, neuroblastoma and pancreatic ductal adenocarcinoma, more preferably the tumor is selected from medulloblastoma and pancreatic ductal adenocarcinoma.

In an embodiment, the 1,2,3,4-tetrahydropyrimidine-5-carboxamide derivative of formula (I), preferably 2,4-dioxo-N-phenyl-3-(2-fluorobenzyl)-1,2,3,4-tetrahydropyrimidine-5-carboxamide, is to be administered at a dose comprised between 2-5 mg/Kg.

In an embodiment, the present invention concerns a pharmaceutical composition comprising at least one 1,2,3,4-tetrahydropyrimidine-5-carboxamide derivative of formula (I) and a pharmaceutically acceptable vehicle.

In an embodiment, the present invention concerns a pharmaceutical composition comprising at least one 1,2,3,4-tetrahydropyrimidine-5-carboxamide derivative of formula (I) and a pharmaceutically acceptable vehicle for use in the treatment of a tumor.

In an embodiment, the at least one 1,2,3,4-tetrahydropyrimidine-5-carboxamide derivative of formula (I) contained in the pharmaceutical composition is selected from:
2,4-dioxo-N-phenyl-3-(2-fluorobenzyl)-1,2,3,4-tetrahydropyrimidine-5-carboxamide (1);
2,4-Dioxo-3-benzyl-N-(3-chlorophenyl)-1,2,3,4-tetrahydropyrimidine-5-carboxamide (2);
2,4-Dioxo-3-(4-fluorobenzyl)-N-(2-fluorophenyl)-1,2,3,4-tetrahydropyrimidine-5-carboxamide (3);
2,4-Dioxo-3-benzyl-N-(2-chlorophenyl)-1,2,3,4-tetrahydropyrimidine-5-carboxamide (4);
2,4-Dioxo-3-benzyl-N-(3,5-dimethoxyphenyl)-1,2,3,4-tetrahydropyrimidine-5-carboxamide (5);
2,4-Dioxo-3-benzyl-N-(4-carboxamidophenyl)-1,2,3,4-tetrahydropyrimidine-5-carboxamide (6);
2,4-Dioxo-3-(4-methoxybenzyl)-N-(2-chlorophenyl)-1,2,3,4-tetrahydropyrimidine-5-carboxamide (7);
2,4-Dioxo-3-(2,4-difluorobenzyl)-N-phenyl-1,2,3,4-tetrahydropyrimidine-5-carboxamide (8);
2,4-Dioxo-3-(2-chlorobenzyl)-N-phenyl-1,2,3,4-tetrahydropyrimidine-5-carboxamide (9);
2,4-Dioxo-3-(4-chlorobenzyl)-N-phenyl-1,2,3,4-tetrahydropyrimidine-5-carboxamide (10);
2,4-Dioxo-3-(2,4-dichlorobenzyl)-N-phenyl-1,2,3,4-tetrahydropyrimidine-5-carboxamide (11);
2,4-Dioxo-3-(3-fluoropyridin-4-ylmethyl)-N-phenyl-1,2,3,4-tetrahydropyrimidine-5-carboxamide (12);
2,4-Dioxo-3-(pyridin-4-ilmethyl)-N-phenyl-1,2,3,4-tetrahydropyrimidine-5-carboxamide (13);
2,4-Dioxo-3-(pyrimidin-4-ilmethyl)-N-phenyl-1,2,3,4-tetrahydropyrimidine-5-carboxamide (14);
2,4-Dioxo-3-(2-fluorobenzyl)-N-(4-fluorophenyl)-1,2,3,4-tetrahydropyrimidine-5-carboxamide (15);
2,4-Dioxo-3-(4-fluorobenzyl)-N-(4-fluorophenyl)-1,2,3,4-tetrahydropyrimidine-5-carboxamide (16);
2,4-Dioxo-3-(2,4-difluorobenzyl)-N-(4-fluorophenyl)-1,2,3,4-tetrahydropyrimidine-5-carboxamide (17);
2,4-Dioxo-3-(benzyl)-N-(2-chlorophenyl)-1,2,3,4-tetrahydropyrimidine-5-carboxamide (18);
2,4-Dioxo-3-(2-fluorobenzyl)-N-(4-chlorophenyl)-1,2,3,4-tetrahydropyrimidine-5-carboxamide (19);
2,4-Dioxo-3-(2-fluorobenzyl)-N-(4-methylphenyl)-1,2,3,4-tetrahydropyrimidine-5-carboxamide (20);
2,4-Dioxo-3-(2-fluorobenzyl)-N-(4-aminophenyl)-1,2,3,4-tetrahydropyrimidine-5-carboxamide (21);
2,4-Dioxo-3-(2-fluorobenzyl)-N-(pyridin-4-yl)-1,2,3,4-tetrahydropyrimidine-5-carboxamide (22);
2,4-Dioxo-3-(2-fluorobenzyl)-N-(pyridin-2-yl)-1,2,3,4-tetrahydropyrimidine-5-carboxamide (23);
2,4-Dioxo-3-(2-fluorobenzyl)-N-(thiazol-2-yl)-1,2,3,4-tetrahydropyrimidine-5-carboxamide (24)
2,4-Dioxo-3-(benzyl)-N-(3,5-dichlorophenyl)-1,2,3,4-tetrahydropyrimidine-5-carboxamide (25).

In an embodiment, the at least one 1,2,3,4-tetrahydropyrimidine-5-carboxamide derivative of formula (I) contained in the pharmaceutical composition is selected from:
2,4-dioxo-N-phenyl-3-(2-fluorobenzyl)-1,2,3,4-tetrahydropyrimidine-5-carboxamide (1);
2,4-Dioxo-3-benzyl-N-(2-chlorophenyl)-1,2,3,4-tetrahydropyrimidine-5-carboxamide (4);
2,4-Dioxo-3-benzyl-N-(4-carboxamidophenyl)-1,2,3,4-tetrahydropyrimidine-5-carboxamide (6);
2,4-Dioxo-3-(4-methoxybenzyl)-N-(2-chlorophenyl)-1,2,3,4-tetrahydropyrimidine-5-carboxamide (7).

In an embodiment, the pharmaceutical composition contains the at least one 1,2,3,4-tetrahydropyrimidine-5-carboxamide derivative of formula (I), preferably 2,4-dioxo-N-phenyl-3-(2-fluorobenzyl)-1,2,3,4-tetrahydropyrimidine-5-carboxamide, at a concentration comprised between 0.001 and 10 mM, preferably 0.01 and 1 mM.

In an embodiment, the present invention concerns the use of a 1,2,3,4-tetrahydropyrimidine-5-carboxamide derivative of formula (I) for the manufacture of a medicament for the treatment of a tumor.

The present disclosure also discloses a method for treating a patient suffering from a tumor comprising the administration to the patient in need thereof of at least one 1,2,3,4-tetrahydropyrimidine-5-carboxamide derivative of formula (I) in an amount sufficient to carry out said treatment.

The current availability of libraries of chemicals with known molecular structure is allowing novel molecular modeling approaches (e.g. molecular docking, pharmacophore modeling) aimed at the identification of candidate molecules for therapies. Using these approaches, the present inventor has identified some candidate molecules to be tested for the ability to inhibit LDH activity.

First of all, the inhibition of the enzyme by oxamate, a well-known LDH inhibitor, was measured-standardized. LDH activity assay was performed on a commercial preparation of the enzyme (Sigma, pyruvic kinase free) with a spectrophotometric assay (at 340 nm), according to the method described by Bergmeyer H. U, Methods of enzymatic analysis, Academic Press, N.Y., 1965 p. 986.

To explore small molecule candidates for the inhibition of LDH, the molecular model of LDH (crystallographic structure from the Protein Data Bank, pdb code 5W8K—Rai G., Brimacombe K. R., Maloney D. J. et al., J Med Chem (2017) 60:9184-9204) was used and the enzyme catalytic task was explored for the interaction with both oxamate and molecules theoretically selected from commercial libraries. To refine the selection, the molecular docking and pharmacophoric study were committed to the Chemical Pharmaceutical Department of Universita degli Studi di Roma “La Sapienza”, laboratory directed by dr. Romano Silvestri.

For the molecular modelling the MacPro dual 2.66 GHz Xeon operative system Ubuntu 14 LTS was used. For the docking the PLANTS software (Korb O, Stützle T, Exner T E, J. Chem. Inf. Model. (2009) 49:84-96) was used with a training set from commercial libraries (www.Maybridge.com, www.SPECS.net and www.Lifechemicals.com) and the candidate molecules were selected according the drug-like rules of Lipinski (Lipinski C A, Lombardo F, Dominy B W, Feeney P J, Adv. Drug Deliv. Rev. (2001) 46:3-26).

From the molecular docking and pharmacophoric study, the present inventor identified derivatives of the 1,2,3,4-tetrahydropyrimidine-5-carboxamide scaffold as novel antitumor drugs able to reduce tumor growth through inhibition of aerobic glycolysis, preferably through the inhibition of the lactate dehydrogenase enzyme.

TABLE 1
Relative inhibitory activity of selected compounds^{a}

| | Growth | LDH |
|---|---|---|
| Compound | Inhibition | Inhibition |
| #1 | *** | *** |
| #2 | * | * |
| #3 | ** | ** |
| #4 | *** | *** |
| #5 | * | * |
| #6 | *** | *** |
| #7 | *** | *** |

^{a}Relative inhibition:
*** strong;
** middle;
* weak.

It was chosen as candidate for tumor cells growth inhibition the molecule 2,4-dioxo-N-phenyl-3-(2-fluorobenzyl)-1,2,3,4-tetrahydropyrimidine-5-carboxamide (compound #1, also named IPI/01.LF). The molecular fitness confirmed that the selected molecule is appropriate for an inhibitory interaction with the catalytic site of the LDH enzyme thanks to a high complementarity of such a molecule with the catalytic site of the enzyme. The compound was then tested to verify whether tumor growth was inhibited by its administration. The results (both in vitro and in vivo) were positive as shown below.

To ascertain the anti-cancer activity of compound #1, the in vitro inhibition of tumor cell growth on established cell lines characterized by the glycolytic shift, such as pancreatic and neurological tumors, was tested. First, these cell lines have been incubated with compound #1 and, as a control, with the standard LDH inhibitor oxamate. Both compounds significantly reduced pancreatic tumor cell (Panc1 cells) proliferation, being the IC₅₀ of compound #1 within the micromolar range, while that of oxamate within the millimolar range.

The newly identified compound #1 showed a comparable ability to reduce tumor growth at a concentration >700 times lower than oxamate. Hence, given the potency and the very encouraging in vitro results, compound #1 was also tested in vivo.

For this purpose, a pancreatic-Panc1 tumor was grown in immunodeficient nu/nu mice; when the tumor mass reached about 200 mm³, mice were treated with compound #1 (20 mg/Kg) vs vehicle (corn oil). Under these experimental conditions, after two weeks the tumor mass doubled in control mice, whereas in the treated group tumors did not grow at all. At the end of the experiment, tumor masses were 2.5 fold smaller in treated mice compared to controls. Conversely, the weight of both groups of mice was unchanged.

An additional experiment was made to evaluate the tumor mass regression with a method similar to those used in humans, e.g. by the in vivo visualization of tumor mass. For this purpose, Panc1 cells were stably transduced with lentivirus (5 MOI) expressing the luciferase gene and subcutaneously implanted in immunodeficient Balb/C nude (nu/nu) athymic mice. After injection of luciferin, the tumor mass has been evaluated for progression/regression on a Lumina III In Vivo Imaging System (PerkinElmer). In mice treated with vehicle, tumors exhibited light-signal emission that increased significantly after 17 days, thus indicating a tumor progression. Conversely, in mice treated with the compound #1 the luminescent light signal showed a significant regression over time, further indicating a strong therapeutic efficacy of this compound in animal models bearing a human tumor.

Collectively, the results witnessed herein clearly show that the inhibitor of LDH herein called #1 or IPI/01.LF [i.e. 2,4-dioxo-N-phenyl-3-(2-fluorobenzyl)-1,2,3,4-tetrahydropyrimidine-5-carboxamide] induces cancer regression in mice bearing human tumors without significant side effects (the weight of both the groups of mice, i.e. treated and control mice, was unchanged). Therefore, compound #1 is a novel and very promising molecule to be further evaluated for therapy of human tumors.

### Materials and Methods

Chemistry. All reagents and solvents were handled according to the material safety data sheet of the supplier and were used as purchased without further purification. Organic solutions were dried over anhydrous sodium sulfate. Evaporation of solvents was carried out on a Büchi Rotavapor R-210 equipped with a Büchi V-850 vacuum controller and a Büchi V-700 vacuum pump. Column chromatography was performed on columns packed with silica gel from Merck (70-230 mesh). Silica gel thin layer chromatography (TLC) cards from Merck (silica gel precoated aluminium cards with fluorescent indicator visualizable at 254 nm) were used for TLC. Developed plates were visualized with a Spectroline ENF 260C/FE UV apparatus. Melting points (mp) were determined on a Stuart Scientific SMP1 apparatus and are uncorrected. Infrared (IR) spectra were recorded on a PerkinElmer Spectrum 100 FT-IR spectrophotometer equipped with a universal attenuated total reflectance accessory. IR data were acquired and processed by PerkinElmer Spectrum 10.03.00.0069 software. Band position and absorption ranges are given in cm⁻¹. Proton nuclear magnetic resonance (¹H NMR) spectra were recorded with a Bruker Avance (400 MHz) spectrometer in the indicated solvent, and the corresponding fid files were processed by MestreLab Research SL MestreReNova 6.2.1-769 software. Chemical shifts are expressed in 6 units (ppm) from tetramethylsilane. The purity of tested compounds was checked by high pressure liquid chromatography (HPLC). Purity of tested compounds was found to be >95%. Thermo Fisher Scientific Inc. Dionex UltiMate 3000 HPLC system consisted of an SR-3000 solvent rack, a LPG-3400SD quaternary analytical pump, a TCC-3000SD column compartment, a DAD-3000 diode array detector, and an analytical manual injection valve with a 20 μL loop. Samples were dissolved in acetonitrile (1 mg/mL). HPLC analysis was performed by using a Thermo Fisher Scientific Inc. Acclaim 120 C18 column (5 μm, 4.6 mm×250 mm), at 25±1° C. with an appropriate solvent gradient (acetonitrile/water), flow rate of 1.0 mL/min and signal detector at 206, 230, 254 and 365 nm. Chromatographic data were acquired and processed by Thermo Fisher Scientific Inc. Chromeleon 6.80 SR15 Build 4656 software.

### General Procedure for the Synthesis of Compounds of Formula (I)

The 1,2,3,4-tetrahydropyrimidine-5-carboxamides of formula (I) are synthesized as shown in Scheme 1, below.

(E)-2-cyano-3-(dimethylamino)acrylic acid is obtained by hydrolysis with lithium hydroxide hydrate of ethyl (E)-2-cyano-3-(dimethylamino)acrylate in tetrahydrofuran at 25° C. for 12 hours. Acrylic acid is transformed into the corresponding carboxamide by reaction with an amine in the presence of (benzotriazol-1-yloxytripyrrolidino-phosphonium hexafluorophosphate) (pyBOP), triethylamine in N,N-dimethylformamide at 25° C. for 12 h in an Argon atmosphere. The carboxamide nitrogen is optionally alkylated by reaction with a halide R₃ in the presence of potassium carbonate in acetone at room temperature. This compound is treated with an N(substituted)-urea in boiling ethanol containing 37% hydrochloric acid for 36 hours, with sodium methoxide in methanol for 1.5 hours at reflux, and then with isopentinyl nitrite in N,N-dimethylformamide at 65° C. for minutes to provide N-(substituted)-2,4-dioxo-3-(substituted)-1,2,3,4-tetrahydropyrimidine-5-carboxamide. Alkylation to Nᵢ is carried out with a halide R₄ in the presence of potassium carbonate in acetone at room temperature. Residues R₁-R₄ have the meanings indicated above.

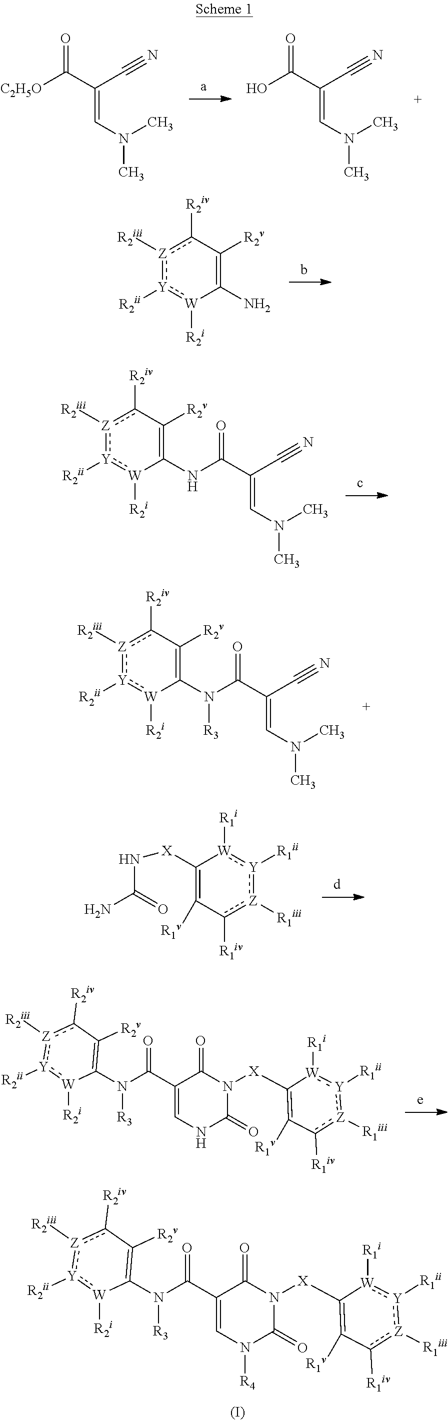

Reagents and reaction conditions: (a) LiOH·H₂O, THF/H₂O, 25° C., 12 h; (b) aniline, pyBOP, Et₃N, DMIF, 25° C., Ar, 12 h; (c) R₃-halogenide, K₂CO₃, acetone, room temperature, overnight; (d) (i) N-(substituted)-urea, 3700 HCl, ethanol, reflux temperature, 36 h; (ii) NaOMe, MeOH, reflux temperature, 1.5 h; (iii) isopentinyl nitrite, DMF, 65° C., 30 min; (e) R₄-halogenide, K₂CO₃, acetone, room temperature, overnight.

### EXAMPLES

### Example 1. Synthesis of Compounds #1-#7

The compound 2.4-dioxo-N-phenyl-3-(2-fluorobenzyl)-1.2.3.4-tetrahydropyrimidine-5-carboxamide (in the following also IPI/01.LF or #1) was synthesized as shown in Scheme 2 below.

Synthesis of 2-cyano-3-(dimethylamino)acrylic acid. A mixture of ethyl 2-cyano-3-(dimethylamino)acrylate (1 mmol) and lithium hydroxide monohydrate (1 mmol) in water/tetrahydrofuran (1:1, 5 mL) was stirred at room temperature for 12 h under argon stream. The reaction mixture was made acidic with 37% hydrogen chloride aqueous solution (pH≈4-5) and extracted with ethyl acetate, dried and filtered. The evaporation of the solvent furnished the 2-cyano-3-(dimethylamino)acrylic acid, that was used without further purification.

Synthesis of (2-cyano-3-(dimethylamino)-N-arylacrylamide. To a mixture of 2-cyano-3-(dimethylamino)acrylic acid (1 mmol), the appropriate aniline (1 mmol), triethylamine (2 mmol) in N,N-dimethylformamide (2 mL) was added (benzotriazol-1-yloxy)tripyrrolidinophosphonium hexafluorophosphate (1 mmol). The reaction mixture was stirred at room temperature for 12 h under argon stream, diluted with water, extracted with ethyl acetate, dried and filtered. The evaporation of the solvent gave the desired (2-cyano-3-(dimethylamino)-N-arylacrylamide, that was used without further purification.

Synthesis of 3-benzyl-2,4-dioxo-N-phenyl-1,2,3,4-tetrahydropyrimidine-5-carboxamide. A mixture of (2-cyano-3-(dimethylamino)-N-arylacrylamide (1 mmol) and the appropriate benzyl urea (1 mmol) in ethanol (2 mL) and 37% hydrogen chloride aqueous solution (0.3 mL) was refluxed for 36 h, cooled, and the solvent removed under reduced pressure. The crude 3-(3-benzylureido)-2-cyano-N-arylacrylamide was used without further purification. To a solution of sodium methoxide in methanol, prepared from sodium (1 mmol) and methanol (5 ml) and ethanol (5 ml), the appropriate 3-(3-benzylureido)-2-cyano-N-arylacrylamide (1 mmol) in methanol (15 ml) was added. The reaction mixture was heated at reflux for 1.5 h and cooled. The solvent was removed under reduced pressure to give the desired 6-amino-1-benzyl-2-oxo-N-aryl-1,2-dihydropyrimidine-5-carboxamide that was used without further purification. To a solution of the appropriate 6-amino-1-benzyl-2-oxo-N-aryl-1,2-dihydropyrimidine-5-carboxamide (1 mmol) in anhydrous N,N-dimethylformamide (3 ml) maintained at 65° C., isopentylnitrite (1.5 mmol) in anhydrous N,N-dimethylformamide (3 ml) was added over 10 min. The mixture was stirred for 30 min. The solvent was removed under reduced pressure to give a solid that was purified by column chromatography (silica gel, chloroform:methanol=95:5) to give the desired 3-benzyl-2,4-dioxo-N-phenyl-1,2,3,4-tetrahydropyrimidine-5-carboxamide.

### 2,4-dioxo-3-(2-Fluorobenzyl)-N-phenyl-1,2,3,4-tetrahydropyrimidine-5-carboxamide (#1)

Yield 68%, mp 234-236° C. (from ethanol). ¹H NMR (DMSO-d₆): δ 5.12 (s, 2H), 10.83 (s, 1H), 7.08-7.16 (m, 2H), 7.20-7.24 (m, 2H), 7.30-7.36 (m, 3H), 7.66 (d, J=7.7 Hz, 2H), 8.39 (s, 1H), 10.83 (br s, disappeared after treatment with deuterium oxide, 1H), 12.37 ppm (br s, disappeared after treatment with deuterium oxide, 1H). IR: v 1686 and 3089 cm⁻¹.

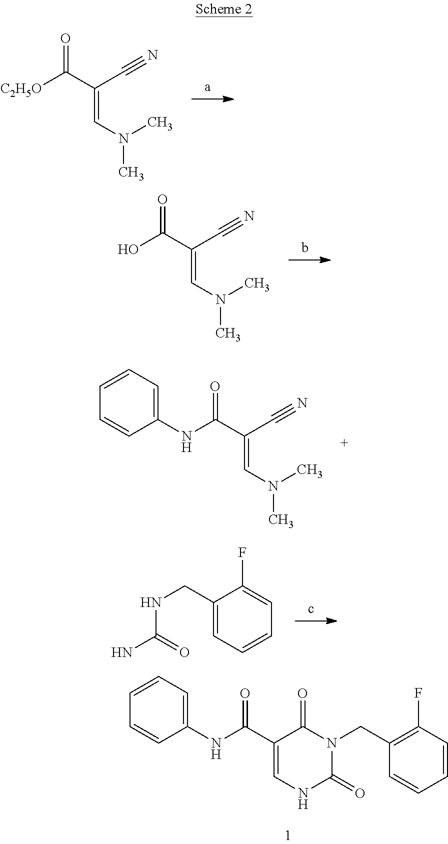

Reagents and reaction conditions: (a) LiOH·H₂O, THF/H₂O, 25° C., 12 h; (b) aniline, pyBOP, Et₃N, DMF, 25° C., Ar, 12 h; (c) (i) N-(2-fluorobenzyl)urea, 37% HCl, ethanol, reflux temperature, 36 h; (ii) NaOMe, MeOH, reflux temperature, 1.5 h; (iii) isopentinyl nitrite, DMF, 65° C., 30 min.

Compounds #2-#7 were Synthesized According to Scheme 2.

### 3-Benzyl-N-(3-chlorophenyl)-2,4-dioxo-, 2,3,4-tetrahydropyrimidine-5-carboxamide (#2)

Yield 76%, mp 208-210° C. (from ethanol). ¹H NMR (DMSO-d₆): δ 5.07 (s, 2H), 7.15-7.17 (m, 1H), 7.25-7.29 (m, 1H), 7.33-7.38 (m, 5H), 7.47-7.50 (m, 1H), 7.96 (t, J=2.1 Hz, 1H), 8.37 (s, 1H), 10.10 (br s, disappeared after treatment with deuterium oxide, 1H), 12.38 ppm (br s, disappeared after treatment with D₂0, 1H). IR: v 1587 and 3087 cm⁻¹.

### 3-(4-fluorobenzyl)-N-(2-fluorophenyl)-2,4-dioxo-1,2,3,4-tetrahydropyrimidine-5-carboxamide (#3)

Yield 70%, mp 204-206° C. (from ethanol). ¹H NMR (DMSO-d₆): δ 5.04 (s, 2H), 7.10-7.23 (m, 4H), 7.29-7.34 (m, 1H), 7.38-7.41 (m, 2H), 8.36-8.41 (m, 2H), 11.23 (br s, disappeared after treatment with deuterium oxide, 1H), 12.40 ppm (br s, disappeared after treatment with deuterium oxide, 1H). IR: v 1616 and 3086 cm⁻¹.

### 3-Benzyl-N-(2-chlorophenyl)-2,4-dioxo-1,2,3,4-tetrahydropyrimidine-5-carboxamide (#4)

Yield 73%, mp 228-230° C. (from ethanol). ¹H NMR (DMSO-d₆): δ 5.08 (s, 2H), 7.12-7.16 (m, 1H), 7.25-7.39 (m, 6H), 7.54 (dd, J=1.4 and 6.6 Hz, 1H), 8.41 (s, 1H), 8.49 (dd, J=6.8 and 11.6 Hz, 1H), 11.39 (br s, disappeared after treatment with deuterium oxide, 1H), 12.40 ppm (br s, disappeared after treatment with deuterium oxide, 1H). IR: v 1582 and 3074 cm⁻¹.

### 3-Benzyl-N-(2,4-dimethoxyphenyl)-2,4-dioxo-1,2,3,4-tetrahydropyrimidine-5-carboxamide (#5)

Yield 79%, mp 232-234° C. (from ethanol). ¹H NMR (DMSO-d₆, 400 MHz): δ 3.76 (s, 3H), 3.86 (s, 3H), 5.07 (s, 2H), 6.51 (dd, J=2.7 and 6.2 Hz, 1H), 6.66 (d, J=2.6 Hz, 1H), 7.25-7.36 (m, 5H), 8.26-8.33 (m, 2H), 11.00 (br s, disappeared after treatment with deuterium oxide, 1H), 12.23 ppm (br s, disappeared after treatment with deuterium oxide, 1H). IR: v 1671 and 3196 cm⁻¹.

### 3-Benzyl-N-(4-carbamoylphenyl)-2,4-dioxo-1,2,3,4-tetrahydropyrimidine-5-carboxamide (#6)

Yield 65%, mp>320° C. (from ethanol). ¹H NMR (DMSO-d₆): δ 5.07 (s, 2H), 7.25-7.29 (m, 2H), 7.33-7.34 (m, 4H), 7.72-7.75 (m, 2H), 7.86-7.89 (m, 3H), 8.38 (s, 1H), 11.06 (br s, disappeared after treatment with deuterium oxide, 1H), 12.37 ppm (br s, disappeared after treatment with deuterium oxide, 1H). IR: v 1604 and 3171 cm⁻¹.

### 3-Benzyl-N-(4-carbamoylphenyl)-2,4-dioxo-1,2,3,4-tetrahydropyrimidine-5-carboxamide (#7)

Yield 71%, mp 220-222° C. (from ethanol). ¹H NMR (DMSO-d₆): δ 3.73 (s, 3H), 5.00 (s, m2H), 6.89 (d, J=8.6 Hz, 2H), 7.12-7.16 (m, 1H), 7.29 (d, J=8.6 Hz, 2H), 7.34-7-39 (m, 1H), 7.54 (dd, J=1.4 and 8.0 Hz, 1H), 8.38 (s, 1H), 8.48 (dd, J=1.5 and 8.3 Hz, 1H), 11.41 (br s, disappeared after treatment with deuterium oxide, 1H), 12.36 ppm (br s, disappeared after treatment with deuterium oxide, 1H). IR: v 1686 and 3075 cm⁻¹.

### Example 2. Oxamate Inhibits Aerobic Glycolysis and Tumor Growth

The experiments of tumor growth inhibition have been programmed by the inventor and partially committed to an experienced team of the Department of Molecular Medicine of Universita degli Studi di Roma “La Sapienza” (under the supervision of Gianluca Canettieri, M.D.).

The classical in vitro test to check the lowering of tumor growth through the inhibition of the aerobic glycolysis is performed with oxamate (a well known LDH inhibitor).

The inventor used tumor cells of medulloblastoma/MB Sonic HedgeHog/SHH (SHH MB), a tumor of the cerebellum due to mutations activating the Hedgehog (HH) signaling pathway (Di Magno L, Coni S, Di Marcotullio L, Canettieri G. Digging a hole under Hedgehog: downstream inhibition as an emerging anticancer strategy Biochim Biophys Acta (Review) (2015) 1856: 62-72; Di Magno L, Manni S, Di Pastena F, Coni S, Macone A, Cairoli S et al. Phenformin Inhibits Hedgehog-Dependent Tumor Growth through a Complex I-Independent Redox Corepressor Module Cell reports (2020) 30:1735-1752). The choice of this tumor was due to the aberrant activation of the Sonic Hedgehog pathway, which causes a reprogramming of energy metabolism toward glycolysis (Di Magno L, Manzi D, D'Amico D, Coni S, Macone A, Infante P et al. Druggable glycolytic requirement for Hedgehog-dependent neuronal and medulloblastoma growth Cell Cycle (2014) 13:3404-3413) by activating specific transcriptional targets. For this reason, SHH MB cells show a marked vulnerability to inhibitors of glycolysis, such as dichloroacetate (DCA) or bromopyruvate, chemicals, which cannot be used in vivo because of their toxicity.

Med1-MB cells (a mouse medulloblastoma cell line kindly provided by Dr Yoon-Jae Cho, Stanford, CA USA and disclosed in Hayden Gephart et al. Journal of Neuro-Oncology (2013) volume 115, pages 161-168) were maintained in DMEM supplemented with 10% FBS and 100 units of penicillin/streptomycin.

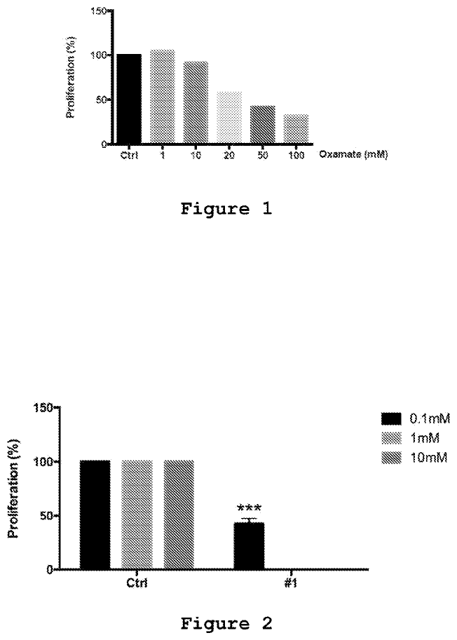

Med1-MB cells were cultured in Dulbecco's modified Eagle medium (DMEM) supplemented with 10% FBS, 1 mM penicillin-streptomycin and 1 mM glutamine in a humidified incubator at 37° C. and 5% CO₂. For the in vitro test, to check the lowering of tumor growth through the inhibition of the aerobic glycolysis, 2.5×10³ Med1-MB cells/cm² were seeded in 12-well plate and incubated overnight at 37° C. After 24 hours from seeding, cells were treated with oxamate at different concentrations, as indicated in FIG. 1. After 72 hours, cells were trypsinized and counted by trypan blue exclusion method.

Oxamate is lowering in vitro proliferation of Med1-MB cells at >20 mM, a dose which is toxic in vivo. The results are shown in FIG. 1.

### Example 3. Effect of IPI/01.LF and Derivatives on Tumor Cell Proliferation

To evaluate the effect on tumor growth of compound IPI/01.LF, and derivatives, 1×10⁴ Med1-MB cells were seeded in 12-well plate and incubated overnight at 37° C. in a humidified incubator. After 24 h, cells were treated with increasing concentrations of compounds for 72 h. At the end of the experiment, cell proliferation was assayed by Trypan Blue exclusion method. IC₅₀ values were determined by generating a dose-response curves by non-linear regression. Data are expressed as the mean±SD of three independent experiments, each performed in triplicate.

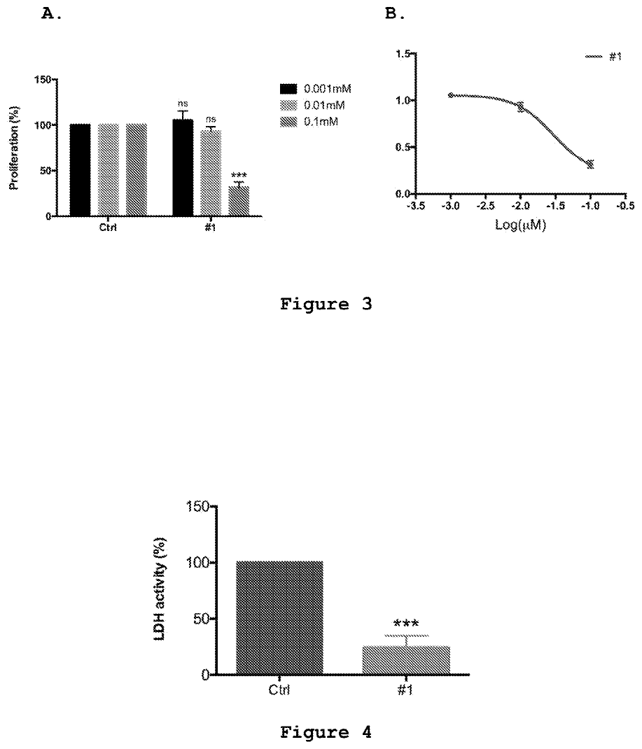

As shown in FIGS. 2 and 3, IPI/01.LF inhibits Med1-IB cell proliferation at micromolar doses, with a calculated IC₅₀ of approximately 0.029 mM.

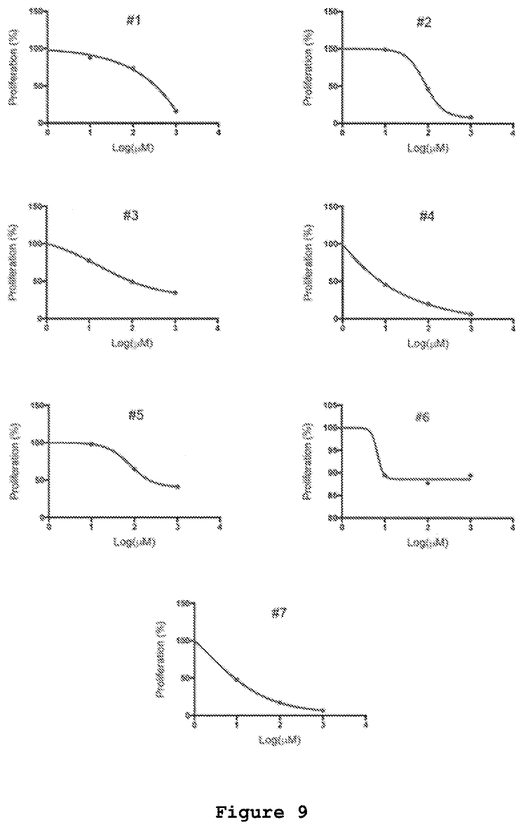

The inhibitory activity of the other 6 derivatives (compounds #2-#7) is indicated in table 1 above and FIG. 9.

The values recorded for IPI/01.LF are approximately 700 times lower than those obtained with oxamate, the reference inhibitor of the LDHA enzyme (see FIG. 1).

### Example 4. Effect of IPI/01.LF and Derivatives on the Activity of the LDHA Enzyme

To evaluate whether the observed anti-proliferative effect of IPI/01.LF and derivatives was a specific consequence of the inhibitory activity on the LDHA enzyme, the inventor performed enzymatic activity assays measuring the rate of NADH consumption following the addition of pyruvate by spectrophotometry at 340 nM, as previously described [Miskimins W K, Ahn H J, Kim J Y, Ryu S, Jung Y S, Choi J Y. Synergistic anti-cancer effect of phenformin and oxamate. PLoS One. 2014; 9(1):e85576.].

1×10⁵ Med1-MB cells were harvested, suspended in 0.1M KH₂PO₄ (pH 7.2), 2 mM EDTA and 1 mM dithiothreitol (DTT), sonicated in 300 μL assay buffer (50 mmol/L potassium phosphate, pH 7.4), and centrifuged at 10,000 g for 10 minutes at 4° C. The supernatant was added to 50 mM potassium phosphate (pH 7.4), 2 mM pyruvate, and 20 M NADH. Absorbance was measured over 10 minutes at 340 nm using a spectrophotometer.

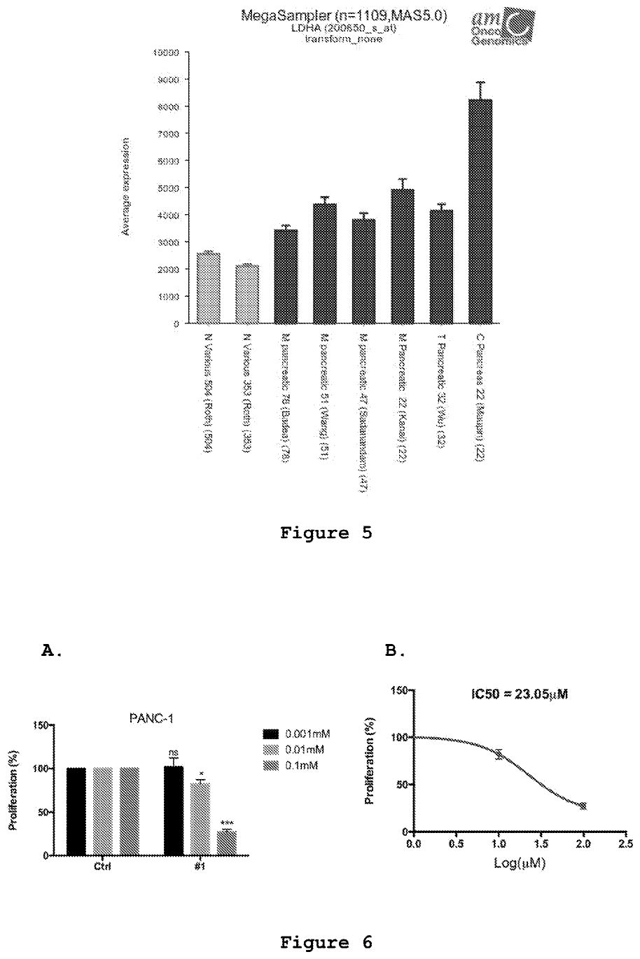

At the dose of 100 μM, IPI/01.LF (compound #1) and derivatives (compounds #2-#7) showed a marked inhibitory effect on LDHA enzymatic activity as shown in FIG. 4 and indicated in table 1 above.

### Example 5. LDHA Gene Expression in Pancreatic Cancer

Gene expression analyzed from the R2 Genomics Analysis and Visualization Platform (http://r2.amc.nl) database indicates that LDHA mRNA expression is significantly higher in human pancreatic adenocarcinoma, a very aggressive tumor with severe prognosis, strongly dependent on metabolic reprogramming in the glycolytic sense by the high expression of LDH gene (Yan L, Raj P, Yao W, Ying H. Glucose Metabolism in Pancreatic Cancer. Cancers (Review) (2019) 11(10):1460) as adaptive response to the request of energy.

Mega Sampler R2 module was used to investigate the expression level of LDHA gene in pancreatic cancer (#6) and normal tissues (#2) datasets stored in the R2 database analysis. The LDHA probeset with the highest average present signal (APS) was selected [(200650_s_at) APS=2246.1(88) Avg=2246.1], and the expression levels of datasets were converted to non transformed values. A one-way ANOVA statistical test was performed, and the resulting graph was plotted in a histogram representation as shown in FIG. 5.

### Example 6. Analysis of Antitumor Activity of IPI/01.LF in Preclinical Models

The inventor tested compound #1-IPI/01.LF on the proliferation of human pancreatic ductal adenocarcinoma (PDAC) Panc1 cells (American Tissue Cell Culture (ATCC, Manassas, VA, USA); product code: ATCC® CRL-1469™) a human tumor cell line showing a high glycolytic rate (Liu L, Gong L, Zhang Y, Li N. Glycolysis in Panc-1 human pancreatic cancer cells is inhibited by everolimus Experimental and therapeutic medicine (2013) 5: 338-342).

PANC-1 cells were maintained in DMEM supplemented with 10% FBS and 100 units of penicillin/streptomycin. 1×10⁵ cells were seeded in 12-well plate and treated with compound IPI/01.LF at the concentrations indicated in FIG. 6. After 72 h, cell proliferation was assayed by Trypan Blue exclusion method. IC₅₀ value was determined by generating a dose-response curves by non-linear regression. Data are expressed as the mean±SD of three independent experiments, each performed in triplicate.

As shown in FIG. 6, compound #1-IPI/01.LF displayed considerable anti-proliferative activity, within the micromolar range, with an IC₅₀ of approximately 0.023 mM, similar to what was observed with Med1-MB cells.

### Example 7. In Vivo Experiments on Compound IPI/01.LF

To evaluate the in vivo therapeutic effects of compound IPI/01.LF studies on immunodeficient nude (nu/nu) mouse models xenografted with the human pancreatic tumor cells Panc1 were performed.

Panc1 cells were stably transduced with 5 MOI of a lentivirus expressing the luciferase gene (pLenti CMV Puro LUC (w168-1)). The lentiviral vector was purchased from Addgene, Watertown, MA 02472 USA (plasmid #17477); Campeau E, et al A versatile viral system for expression and depletion of proteins in mammalian cells PLoS One (2009) 4(8):e6529.

Stably transduced Panc1 cells were subcutaneously implanted in immunocompromised Balb/C nude athymic mice. When the tumors reached the volume of 200 mm³, mice were treated with either 20 mg/kg of compound IPI/01.LF dissolved in corn oil or with corn oil (vehicle) alone and injected intraperitoneally every two days. Tumor growth was monitored with a caliper every other day. Data are expressed as the mean±SD of three independent experiments, each performed in triplicate.

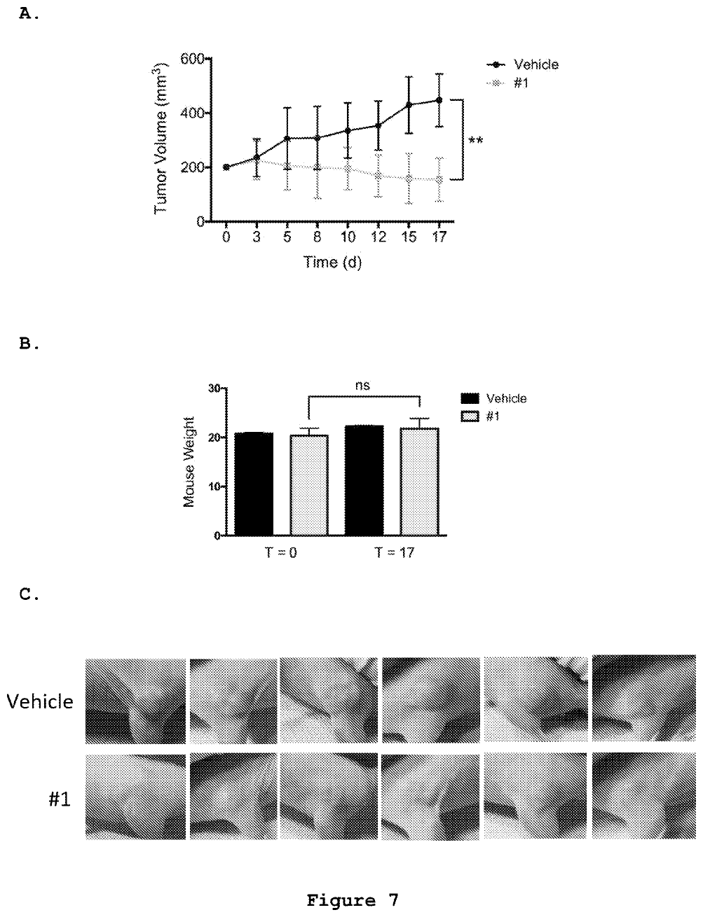

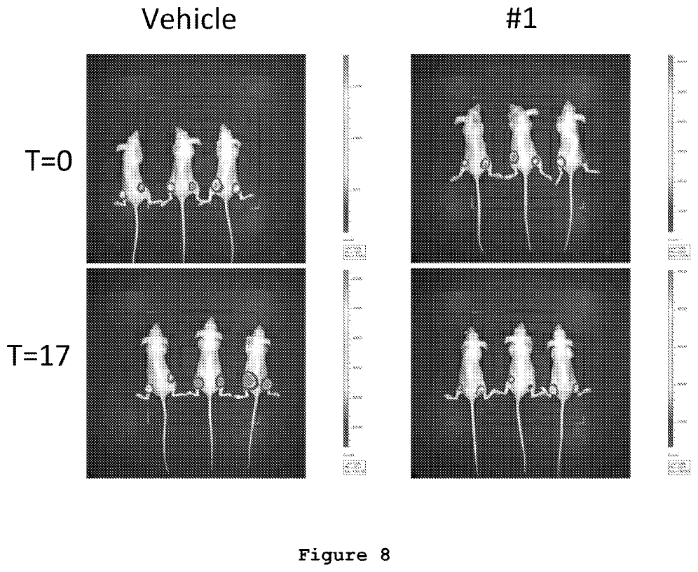

As shown in FIG. 7, tumor growth rate in vivo was significantly lower in mice treated with compound IPI/01.LF compared to control mice treated with vehicle. At the end of treatment, the mean volume of tumors was significantly reduced compared to controls, while the weight of both mice groups was unchanged.

Since the grafted Panc1 tumor cells express the ectopic luciferase reporter gene, tumor luminescence was used to monitor the tumor growth. Luminescence was measured using IVIS Lumina III In Vivo Imaging System (PerkinElmer).

Mice were intraperitoneally injected with luciferin (RediJect D-Luciferin Bioluminescent Substrate, PerkinElmer) and analyzed by photon emission (bioluminescence).

L of luciferin (15 mg/mL stock)/gram of mouse body weight were injected in the lower left abdominal quadrant of xenografted Balb/C mice with a 25 gauge needle. After 10 minutes, animals were anesthetized with a mixture of oxygen gas and vaporized isoflurane. Once the animals were fully anesthetized, they were positioned within the animal imaging cassette for image acquisition. Data were analyzed utilizing the LivingImage software. Image acquisition was performed at the beginning (T=0) and at the end (T=17) of the experiment and body weights (g) of mice at time of sacrifice were determined.

In mice treated with vehicle alone, tumors exhibited light-signal emission that increased significantly over time (FIG. 8). Conversely, in mice treated with 20 mg/Kg IPI/01.LF the luminescent signal showed a significant regression over time, further indicating the therapeutic efficacy of the compound in animal models bearing a human tumor.

Overall, these data demonstrate that LDH inhibitor IPI/01.LF has a marked specific inhibitory activity on the LDH enzyme and a robust antitumor effect in vitro and in vivo.

1. A 1,2,3,4-tetrahydropyrimidine-5-carboxamide derivative of formula (I): 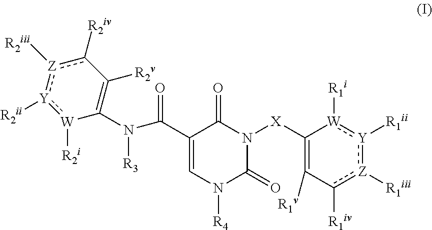
wherein
R₁ⁱ, R₁ⁱⁱ, R₁ⁱⁱⁱ, R₁^{v}, R₂ⁱ, R₂ⁱⁱ, R₂ⁱⁱⁱ, R₂^{v} are independently selected from: none, hydrogen, C₁₋₆ alkyl, C₃₋₁₀ cycloalkyl, C₂₋₆ alkenyl, C₂₋₆ alkynyl, C₁₋₆ alkoxy, C₂₋₆ alkoxycarbonyl, 3-10 member heterocycloalkyl, halogen, —CN, NH₂, OH, NO₂, OR₇, NHR₈, NR₇R₈ and NHCOR₇;
R₃ and R₄ are independently selected from: hydrogen, C₁₋₆ alkyl, C₃₋₁₀ cycloalkyl, C₂₋₆ alkenyl, C₂₋₆ alkynyl, C₁₋₆ alkoxy, C₂₋₆ alkoxycarbonyl, 3-10-member heterocycloalkyl, halogen, CN, NH₂, OH, and NO₂;
R₇ and R₈ are independently selected from: C₁₋₆ alkyl, C₃₋₁₀ cycloalkyl, C₂₋₆ alkenyl, C₂₋₆ alkynyl, C₁₋₆ alkoxy, C₂₋₆ alkoxycarbonyl, 3-10 member heterocycloalkyl, and a substituted or unsubstituted aromatic ring Ar₁;
X is selected from: CH₂, C(═O), S, SO₂, SO, and none; and
W, Y and Z are independently selected from: C, N, C(═O), C(═S);
a pharmaceutically acceptable salt, hydrated salt, polymorph, racemate, diastereoisomer or enantiomer thereof,
with the exception of:
2,4-Dioxo-3-(2-fluorobenzyl)-N-phenyl-1,2,3,4-tetrahydropyrimidine-5-carboxamide,
2,4-Dioxo-3-(2-chlorobenzyl)-N-phenyl-1,2,3,4-tetrahydropyrimidine-5-carboxamide,
2,4-Dioxo-3-(2-fluorobenzyl)-N-(4-fluorophenyl)-1,2,3,4-tetrahydropyrimidine-5-carboxamide,
2,4-Dioxo-3-(2-fluorobenzyl)-N-(4-chlorophenyl)-1,2,3,4-tetrahydropyrimidine-5-carboxamide,
2,4-Dioxo-3-(2-fluorobenzyl)-N-(4-methylphenyl)-1,2,3,4-tetrahydropyrimidine-5-carboxamide,
2,4-Dioxo-3-(2-fluorobenzyl)-N-(pyridin-4-yl)-1,2,3,4-tetrahydropyrimidine-5-carboxamide,
2,4-Dioxo-3-(2-fluorobenzyl)-N-(pyridin-2-yl)-1,2,3,4-tetrahydropyrimidine-5-carboxamide,
2,4-Dioxo-3-(2-fluorobenzyl)-N-(thiazol-2-yl)-1,2,3,4-tetrahydropyrimidine-5-carboxamide.

2. The 1,2,3,4-tetrahydropyrimidine-5-carboxamide derivative according to claim 1, wherein R₁ⁱ, R₁ⁱⁱ, R₁ⁱⁱⁱ, R₂^{v}, R₂ⁱ, R₂ⁱⁱ, R₂ⁱⁱⁱ, R₂^{v}, R₃, and R₄ are independently selected from: hydrogen, C₁₋₆ alkyl, halogen.

3. The 1,2,3,4-tetrahydropyrimidine-5-carboxamide derivative according to claim 1, wherein R₃ and R₄ are independently selected from: hydrogen, C₁₋₆ alkyl, and C₃₋₁₀ cycloalkyl.

4. The 1,2,3,4-tetrahydropyrimidine-5-carboxamide derivative according to claim 1, wherein R₃ and R₄ are hydrogen.

5. The 1,2,3,4-tetrahydropyrimidine-5-carboxamide derivative according to claim 1, wherein R₇ and R₈ are independently selected from: C₁₋₆ alkyl, and a substituted or unsubstituted aromatic ring Ar₁.

6. The 1,2,3,4-tetrahydropyrimidine-5-carboxamide derivative according to claim 1, wherein, X is selected from: CH₂, C(═O).

7. The 1,2,3,4-tetrahydropyrimidine-5-carboxamide derivative according to claim 1, wherein the aromatic ring Ar₁ is selected from: phenyl, benzyl, phenoxy, benzyloxy, naphthyl, naphthyloxy, biphenyl, and heterocycle.

8. The 1,2,3,4-tetrahydropyrimidine-5-carboxamide derivative according to claim 1, wherein the 1,2,3,4-tetrahydropyrimidine derivative of formula (I) is selected from:
2,4-Dioxo-3-benzyl-N-(3-chlorophenyl)-1,2,3,4-tetrahydropyrimidine-5-carboxamide (2);
2,4-Dioxo-3-(4-fluorobenzyl)-N-(2-fluorophenyl)-1,2,3,4-tetrahydropyrimidine-5-carboxamide (3);
2,4-Dioxo-3-benzyl-N-(2-chlorophenyl)-1,2,3,4-tetrahydropyrimidine-5-carboxamide (4);
2,4-Dioxo-3-benzyl-N-(3,5-dimethoxyphenyl)-1,2,3,4-tetrahydropyrimidine-5-carboxamide (5);
2,4-Dioxo-3-benzyl-N-(4-carboxamidophenyl)-1,2,3,4-tetrahydropyrimidine-5-carboxamide (6);
2,4-Dioxo-3-(4-methoxybenzyl)-N-(2-chlorophenyl)-1,2,3,4-tetrahydropyrimidine-5-carboxamide (7);
2,4-Dioxo-3-(2,4-difluorobenzyl)-N-phenyl-1,2,3,4-tetrahydropyrimidine-5-carboxamide (8);
2,4-Dioxo-3-(4-chlorobenzyl)-N-phenyl-1,2,3,4-tetrahydropyrimidine-5-carboxamide (10);
2,4-Dioxo-3-(2,4-dichlorobenzyl)-N-phenyl-1,2,3,4-tetrahydropyrimidine-5-carboxamide (11);
2,4-Dioxo-3-(3-fluoropyridin-4-ylmethyl)-N-phenyl-1,2,3,4-tetrahydropyrimidine-5-carboxamide (12);
2,4-Dioxo-3-(pyridin-4-ilmethyl)-N-phenyl-1,2,3,4-tetrahydropyrimidine-5-carboxamide (13);
2,4-Dioxo-3-(pyrimidin-4-ilmethyl)-N-phenyl-1,2,3,4-tetrahydropyrimidine-5-carboxamide (14);
2,4-Dioxo-3-(4-fluorobenzyl)-N-(4-fluorophenyl)-1,2,3,4-tetrahydropyrimidine-5-carboxamide (16);
2,4-Dioxo-3-(2,4-difluorobenzyl)-N-(4-fluorophenyl)-1,2,3,4-tetrahydropyrimidine-5-carboxamide (17);
2,4-Dioxo-3-(benzyl)-N-(2-chlorophenyl)-1,2,3,4-tetrahydropyrimidine-5-carboxamide (18);
2,4-Dioxo-3-(2-fluorobenzyl)-N-(4-aminophenyl)-1,2,3,4-tetrahydropyrimidine-5-carboxamide (21);
2,4-Dioxo-3-(benzyl)-N-(3,5-dichlorophenyl)-1,2,3,4-tetrahydropyrimidine-5-carboxamide (25).

9. A method of treating a patient suffering from a tumor comprising administering a 1,2,3,4-tetrahydropyrimidine-5-carboxamide derivative of formula (I), pharmaceutically acceptable salt, hydrated salt, polymorph, racemate, diastereoisomer or enantiomer thereof according to claim 1 to the patient.

10. The method according to claim 9, wherein the 1,2,3,4-tetrahydropyrimidine derivative is selected from:
2,4-dioxo-N-phenyl-3-(2-fluorobenzyl)-1,2,3,4-tetrahydropyrimidine-5-carboxamide (1);
2,4-Dioxo-3-benzyl-N-(3-chlorophenyl)-1,2,3,4-tetrahydropyrimidine-5-carboxamide (2);
2,4-Dioxo-3-(4-fluorobenzyl)-N-(2-fluorophenyl)-1,2,3,4-tetrahydropyrimidine-5-carboxamide (3);
2,4-Dioxo-3-benzyl-N-(2-chlorophenyl)-1,2,3,4-tetrahydropyrimidine-5-carboxamide (4);
2,4-Dioxo-3-benzyl-N-(3,5-dimethoxyphenyl)-1,2,3,4-tetrahydropyrimidine-5-carboxamide (5);
2,4-Dioxo-3-benzyl-N-(4-carboxamidophenyl)-1,2,3,4-tetrahydropyrimidine-5-carboxamide (6);
2,4-Dioxo-3-(4-methoxybenzyl)-N-(2-chlorophenyl)-1,2,3,4-tetrahydropyrimidine-5-carboxamide (7);
2,4-Dioxo-3-(2,4-difluorobenzyl)-N-phenyl-1,2,3,4-tetrahydropyrimidine-5-carboxamide (8);
2,4-Dioxo-3-(2-chlorobenzyl)-N-phenyl-1,2,3,4-tetrahydropyrimidine-5-carboxamide (9);
2,4-Dioxo-3-(4-chlorobenzyl)-N-phenyl-1,2,3,4-tetrahydropyrimidine-5-carboxamide (10);
2,4-Dioxo-3-(2,4-dichlorobenzyl)-N-phenyl-1,2,3,4-tetrahydropyrimidine-5-carboxamide (11);
2,4-Dioxo-3-(3-fluoropyridin-4-ylmethyl)-N-phenyl-1,2,3,4-tetrahydropyrimidine-5-carboxamide (12);
2,4-Dioxo-3-(pyridin-4-ilmethyl)-N-phenyl-1,2,3,4-tetrahydropyrimidine-5-carboxamide (13);
2,4-Dioxo-3-(pyrimidin-4-ilmethyl)-N-phenyl-1,2,3,4-tetrahydropyrimidine-5-carboxamide (14);
2,4-Dioxo-3-(2-fluorobenzyl)-N-(4-fluorophenyl)-1,2,3,4-tetrahydropyrimidine-5-carboxamide (15);
2,4-Dioxo-3-(4-fluorobenzyl)-N-(4-fluorophenyl)-1,2,3,4-tetrahydropyrimidine-5-carboxamide (16);
2,4-Dioxo-3-(2,4-difluorobenzyl)-N-(4-fluorophenyl)-1,2,3,4-tetrahydropyrimidine-5-carboxamide (17);
2,4-Dioxo-3-(benzyl)-N-(2-chlorophenyl)-1,2,3,4-tetrahydropyrimidine-5-carboxamide (18);
2,4-Dioxo-3-(2-fluorobenzyl)-N-(4-chlorophenyl)-1,2,3,4-tetrahydropyrimidine-5-carboxamide (19);
2,4-Dioxo-3-(2-fluorobenzyl)-N-(4-methylphenyl)-1,2,3,4-tetrahydropyrimidine-5-carboxamide (20);
2,4-Dioxo-3-(2-fluorobenzyl)-N-(4-aminophenyl)-1,2,3,4-tetrahydropyrimidine-5-carboxamide (21);
2,4-Dioxo-3-(2-fluorobenzyl)-N-(pyridin-4-yl)-1,2,3,4-tetrahydropyrimidine-5-carboxamide (22);
2,4-Dioxo-3-(2-fluorobenzyl)-N-(pyridin-2-yl)-1,2,3,4-tetrahydropyrimidine-5-carboxamide (23);
2,4-Dioxo-3-(2-fluorobenzyl)-N-(thiazol-2-yl)-1,2,3,4-tetrahydropyrimidine-5-carboxamide (24)
2,4-Dioxo-3-(benzyl)-N-(3,5-dichlorophenyl)-1,2,3,4-tetrahydropyrimidine-5-carboxamide (25).

11. The use method according to claim 9, wherein the tumor is selected from brain and pancreatic tumors.

12. The method according to claim 9, wherein the tumor is selected from medulloblastoma, neuroblastoma and pancreatic ductal adenocarcinoma.

13. A pharmaceutical composition comprising at least one 1,2,3,4-tetrahydropyrimidine-5-carboxamide derivative of formula (I), pharmaceutically acceptable salt, hydrated salt, polymorph, racemate, diastereoisomer or enantiomer thereof according to claim 1, and a pharmaceutically acceptable vehicle.
